(19) United States
(12) Patent Application Publication
(10) Pub. No.: US 2016/0206237 A1
(43) Pub. Date: Jul. 21, 2016

(54) SYSTEM AND METHOD FOR MONITORING PRESSURE DISTRIBUTION OVER A PRESSURE-DETECTION MAT WITH DISCONTINUITIES

(71) Applicant: Enhanced Surface Dynamics, Inc., Palo Alto, CA (US)

(72) Inventors: Ronen Friedlander, Rehovot (IL); Yan Rosh, Netanya (IL)

(21) Appl. No.: 15/000,882

(22) Filed: Jan. 19, 2016

Related U.S. Application Data
(60) Provisional application No. 62/104,767, filed on Jan. 18, 2015.

(51) Int. Cl. A61B 5/00; A61B 5/103

(52) U.S. Cl. CPC A61B 5/447; A61B 5/1036; A61B 5/742

(57) ABSTRACT

A system and method for a pressure-detection mat used in an operating table for monitoring pressure of a subject undergoing a medical procedure and preventing the development of pressure ulcers. The disclosure relates to pressure distribution monitoring over an operating table mattress having at least one discontinuity bridged by using bridging wires to connect a first segment and a second segment of a conducting strip created by the discontinuity. The pressure distribution may be presented on a display unit in a normalized manner to provide indications for the operating team.

### CROSS-REFERENCE TO RELATED APPLICATIONS

This application claims the benefit of U.S. provisional application Ser. No. 62/104,767 filed Jan. 18, 2015, the disclosure of which is hereby incorporated in its entirety by reference herein.

### TECHNICAL FIELD

The disclosure herein relates to a pressure monitoring system and method for preventing the development of pressure wounds during a medical procedure. In particular, the disclosure relates to pressure distribution monitoring over an operating theatre table mattress having at least one bridged discontinuity to reduce the potential development of pressure wounds such as decubitus ulcers or bedsores.

### BACKGROUND

Pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 2.5 million hospitalized patients are estimated to have pressure ulcers each year (Source: Medicare website 2009). It's been further reported of 257,412 cases of preventable pressure ulcers as secondary diagnoses. Although easily preventable and treatable if found early, pressure ulcers are painful, and treatment is both difficult and expensive. Further, undergoing a medical procedure in an operating theatre may make things worse. Heizenroth Pa., in his article of “Positioning the Patient for Surgery” stated that approximately 1.5 million hospitalized patients in the U.S. will develop pressure ulcers with about 30% to 40% of these ulcerations starting in the operating theatre. Research shows that 8.5% of all patients having surgical procedures that last longer than three hours develop pressure ulcers.

Operating theatre technology is rapidly advancing, especially due to the advancement in surgical equipment and enormous possibilities offered by digital technology helping to advance digital surgical technology. Features, such as touch screen capability, instant access to images, voice activation, high definition pictures and video capture, image playback and the like may be provided. Medical specialties have different demanding requirements for the functioning of the operating room and the surgical theatre has to be appropriate for the various medical fields. In particular, a state-of-the-art operating table is critical to surgery's success for all medical disciplines requiring adjustments to accommodate to different medical procedures from orthopedic to hernia surgery, urological, gynecological to cosmetic surgery and much more. Further, the complexities of medical procedures and the diversities of medical equipment may introduce pressure detection mats for the operating table with discontinuities.

There is therefore a need for a practical technical solution for bridging the discontinuity region and provide a pressure-detection mat answering the needs of operating theatre table for monitoring pressure distribution throughout. The present disclosure addresses this need.

### SUMMARY

According to one aspect of the disclosure, a pressure-detection mat is presented comprising at least one layer of an insulating material sandwiched between a first layer of conducting strips and a second layer of conducting strips, the conducting strips of the first electrode layer and the conducting strips of the second layer overlapping at a plurality of intersections; a first bundle of connecting wires for connecting the conducting strips of the first layer to a control unit; a second bundle of connecting wires for connecting the conducting strips of the second layer to the control unit. The pressure-detection mat may comprise at least one discontinuity such that at least one conducting strip of the first layer is non-continuous and includes at least a first segment and a second segment; and the first segment is connected to the second segment by a bridging wire to provide conductive communication therebetween.

As appropriate, the conducting strip of the first segment comprises a composite of a 20 conductive material and a conductive wire.

As appropriate, the conducting strip of the pressure-detection mat may comprise an array of strip electrodes embedded in the insulating material, each of the strip electrode comprising a plurality of segments of conductive material; a connecting wire in conductive contact with said segments, said connecting wire having a length exceeding the length of the strip electrode such that said connecting wire adopts a sinuous configuration along the strip electrode; and a flexible laminate into which said segments and said connecting wire are embedded; and the first layer and the second layer are orientated such that the strip electrodes of the first layer and the strip electrodes of the second electrode layer overlap at a plurality of intersections.

As appropriate, the discontinuity may have a closed shape within the boundaries of the pressure-detection mat or may be shaped as an open shape within the boundaries of said pressure-detection mat.

Optionally, the bridging wire may be coupled to the insulating material by an insulated fastener. Additionally, the bridging wire may circumvent the discontinuity.

Accordingly, the bridging wire is in conductive contact with the connecting wires of the first segment and the second segment of the conducting strips.

According to another aspect of the disclosure, a pressure-detection surface system is presented comprising a first pressure-detection mat as described hereinabove; a driving unit configured to supply electrical potential selectively to the conducting strips; the control unit wired to the conductive strips and operable to control the driving unit; a processor configured to monitor electrical potential on the conductive strips, to calculate impedance values for each intersection and to determine pressure applied to the intersection; and at least one display unit configured to present a pressure distribution map to at least one caretaker.

Further, the surface detection system may comprise at least a second pressure-detection mat wherein the processor is further configured to normalize the pressure distribution map data recorded by the first pressure-detection mat and the second pressure-detection mat such that a normalized map is presented on the at least one display unit.

It is yet another aspect of the current disclosure to teach a method for manufacturing a pressure detection mat. The method may comprise obtaining at least one first layer comprising a 20 plurality of conducting strips; obtaining at least one second layer comprising a plurality of conducting strips; obtaining at least one layer of an insulating material sandwiched between the first layer and the second layer; providing at least one non-continuous conducting strip including at least a first segment and at least a second segment separated by a discontinuity; and bridging the discontinuity by connecting the first segment and the second segment of said conducting strip 25 with at least one bridging wire.

As appropriate, the method further comprises embedding at least one bridging wire within a flexible laminate into the insulating material.

Still it is another aspect of the present disclosure to teach a method for processing pressure distribution in a pressure-detection surface system, the system comprising a first pressure-detection mat, a driving unit configured to supply electrical potential selectively to conducting strips, a control unit wired to the conductive strips and operable to control the driving unit, a processor configured to monitor electrical potential on the conductive strips, to calculate impedance values for each intersection and to determine pressure applied to the intersection and at least one display unit configured to present a pressure distribution map, the method comprising: identifying surface sections of the pressure-detection surface system; receiving pressure values determined to each of the intersection for each surface section; storing the pressure values for each of the intersection in at least one data storage; computing a pressure distribution map for each identified surface section based upon the stored pressure values; normalizing the pressure distribution map for each of the identified surface section; and presenting a normalized pressure distribution view comprising at least one pressure distribution map on at least one display unit.

Accordingly, the first pressure-detection mat comprises at least one layer of an insulating material sandwiched between a first layer of conducting strips and a second layer of conducting strips, the conducting strips of the first electrode layer and the conducting strips of the second layer overlapping at a plurality of intersections; a first bundle of connecting wires for connecting the conducting strips of the first layer to a control unit; a second bundle of connecting wires for connecting the conducting strips of the second layer to the control unit; where the pressure-detection mat comprises at least one discontinuity such that at least one conducting strip of the first layer is non-continuous and includes at least a first segment and a second segment; and where the first segment is connected to said second segment by a bridging wire to provide conductive communication therebetween.

### BRIEF DESCRIPTION OF THE DRAWINGS

For a better understanding of the embodiments and to show how they may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1 is a schematic block diagram of the main components of a pressure monitoring system according to an embodiment;

FIG. 2A-C schematically represents various top views of operating theatre operation tables for different medical procedures, with various number of pressure distribution maps including possible connectivity to a monitoring system;

FIGS. 3A and 3B schematically represent isometric and exploded views of an example of a composite flexible conductive material including conducting wire embedded in a flexible laminate according to another embodiment of a conductive flexible material;

FIG. 4A schematically represents a selectively conducting fabric wired to function as an array of strip electrodes;

FIG. 4B schematically represents a flexible conductive material in which an array of embedded conducting segments are wired to function as an array of strip electrodes;

FIG. 5A schematically represents an exploded isometric view of a particular application of the disclosure in which selectively conducting fabric are configured to serve as electrode layers of a pressure sensing surface;

FIG. 5B schematically represents an exploded isometric view of another embodiment of the pressure sensing surface in which composite flexible conductive materials are configured to serve as the electrode layers;

FIG. 6A schematically represents an isometric embodiment of a three-section anti-decubitus operating table mattress system with a discontinuity region;

FIG. 6B schematically represents an exploded isometric view of an anti-decubitus operating table mattress section detailing the technical bridging solution of the current disclosure;

FIG. 6C schematically represents a top-view embodiment of an anti-decubitus operating table mattress section with a discontinuity region;

FIG. 6D schematically represents an exploded top-view view of an anti-decubitus operating table mattress section detailing the technical bridging solution of the current disclosure;

FIG. 7A shows a possible split view display screen for indicating the current orientation and pressure distribution of subject's body on a three section adjustable surface;

FIG. 7B shows a possible unified display screen for indicating a recommendation orientation plan of subject's body on a three section adjustable surface;

FIG. 8A-D show various pressure distribution maps of various posture representations displayed on a display screen;

FIG. 9 is a flowchart representing selected actions of a method for providing a technical bridging solution for a flexible pressure-detection mat discontinuity of current disclosure; and

FIG. 10 is a flowchart representing selected actions of a method for normalizing pressure distribution map of a subject lying on an operation table.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Aspects of the present disclosure relate to a pressure monitoring system and method for preventing the development of pressure ulcers during medical procedure in an operation theatre. The disclosure relates to pressure distribution monitoring using an operating table with at least one pressure-detection mat having at least one discontinuity, where bridging the discontinuity is a particular feature of the current disclosure, to allow comprehensive monitoring and reduce the potential development of pressure ulcers or bedsores.

Various risk factors may contribute to the formation of pressure ulcers during surgery. Patient immobility throughout the surgery for a lengthy medical procedure has a major impact, thus, monitoring is of utmost importance. When a patient is immobile, the chance of pressure ulcer formation multiplies. Patients undergoing lengthy surgical procedures are at higher risk for ulceration, mainly because the patient may be positioned in a manner causes increased pressure on specific body parts and unable to change position during the procedure. Further, the patient may be subjected to prolonged anesthesia, which leads to absence of sensory perception. Furthermore, positioning involves moving, stabilizing and securing the patient's body to allow for the optimal exposure of the surgical procedure while maintaining physiological functions and keeping patient comfort.

Surgical position may vary according to a medical procedure. Known surgical position is Supine position—where the patient lies on his back; Lithotomy position—used for gynecological, anal, and urological procedures. Upper torso is placed in the supine position, legs are raised and secured, arms are extended; Prone position—patient lies with stomach on the bed. Abdomen can be raised off the bed; Lateral position—also called the side-lying position Patient's abdomen lies flat on the bed and the patient is on his or her side; Trendelenburg position—similar to Supine, but the upper torso is lowered; Reverse Trendelenburg position—similar to Supine position but the upper torso is raised and legs are lowered.

The pressure-detection monitoring system of the current disclosure may include at least one pressure-detection surface, a plurality of sensors configured to detect pressure, at least one orientation sensor configured to detect surface angles, at least one driving unit configured to supply electrical potential to the sensors, at least one control unit configured to control the driving units and receive data from the sensors, at least one processor configured to interpret and analyze the data and at least one display configured to present the data and bridging connections over at least one discontinuity. The system may further include at least one storage unit configured to store data from the control units and processors.

The number of pressure-detection sections of a surface may vary according to the medical procedure and are typically integrated into areas of an operation table

It is noted that the systems and methods of the disclosure herein may not be limited in their application to the details of construction and the arrangement of the components or methods set forth in the description or illustrated in the drawings and examples. The systems and methods of the disclosure may be capable of other embodiments or of being practiced or carried out in various ways.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

### System Configuration:

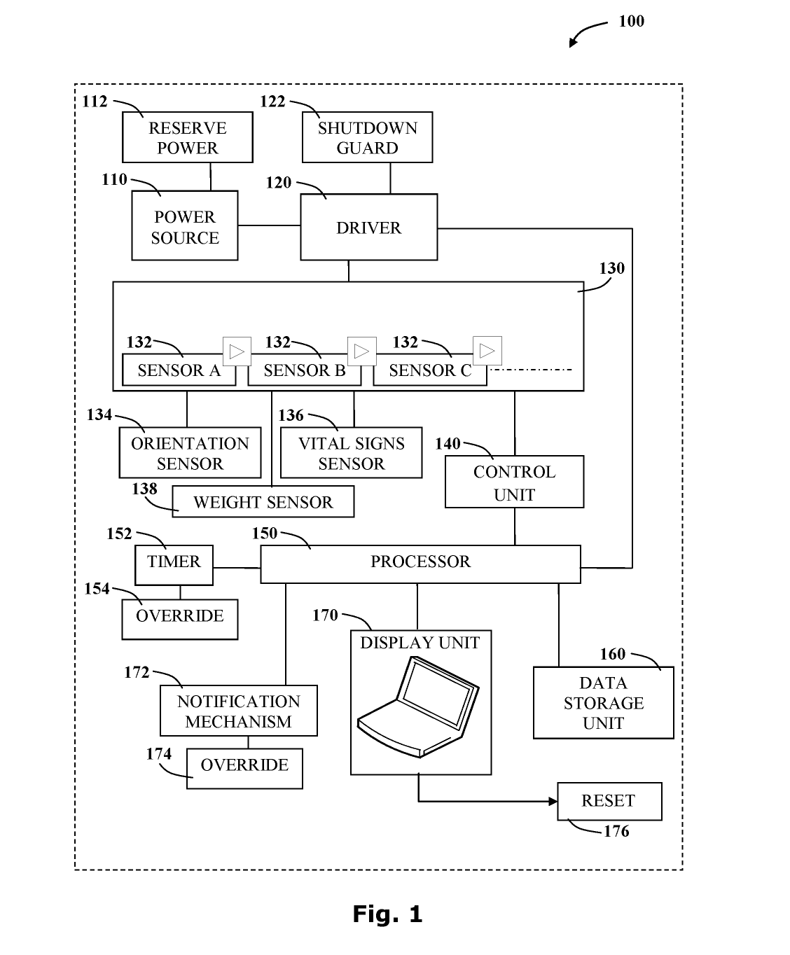

Reference is now made to the block diagram of FIG. 1 showing an embodiment of a pressure monitoring system 100 in an operating theatre. The system 100 may include at least one pressure-detection surface 130 including a plurality of pressure sensors 132 and at least one orientation sensor 134 for each adjustable surface section, a driver 120, and a control unit 140 which may be connected to a power source 110, a processor 150, a data storage unit 160 and a display system 170. Power may be supplied via a power cord connected to a wall outlet, or via battery power, optionally rechargeable. Battery support also allows for movement of the bed without requiring a powering off of the system. As a safety measure and for compliance tracking, caregiver authentication may be required via a shutdown guard 122 to confirm powering off the control unit 140, such as with entry of a caregiver's employee identification number.

In one embodiment, the driver 120 selectively supplies voltage to sensors 132 in the pressure-detection sheet, the processor 150 monitors the potential across the sensors 132, calculates impedance values for each sensor 132, and stores that data in a data storage unit 160. The stored data may be further processed, analyzed, and displayed on a display system 170, such as computer screens, laptops, PDAs, cellular phone screens, printed sheets, integrated LCD screens (e.g. TFT, touch screen) and the like. Although presented in the block diagram of FIG. 1 as separate blocks, the pressure monitoring system 100 may optionally be integrated with existing operating theatre technology, specifically integrated with other monitoring and alerting systems of the operating table to answer specific medical requirements.

### Operating Theatre Tables:

An operating room may be designed and equipped to provide care to patients with a range of conditions, or it may be designed and equipped to provide specialized care to patients with specific conditions. As appropriate, an operating room has special equipment designed around the operating table, such as respiratory and cardiac support, emergency revitalization devices, patient monitoring and various diagnostic tools.

Specifically, operating table technology considers various operating theatre needs such as safety, sterility, simplicity and allowing for technology integration. Safety of patients requires monitoring systems to support multi-functional medical procedures, with pressure detection monitoring and alerting combined with patient repositioning plays an important role. For example, duplicate monitors may be combined into large wall-mounted data information displays, or aspects of the operating room may be controlled from one handheld wireless device and the like.

Operating table may be designed to answer multi-functional surgery needs, such as to allow heavier patients, greater tilting, cantilevering capabilities, greater table top extensions, wiring, vacuum, gas noses incorporated into the table architecture, padding the table top may be made of composite fabric material such as described in the applicant's international patent applications PCT/IB2013/059499 which is incorporated herein by reference.

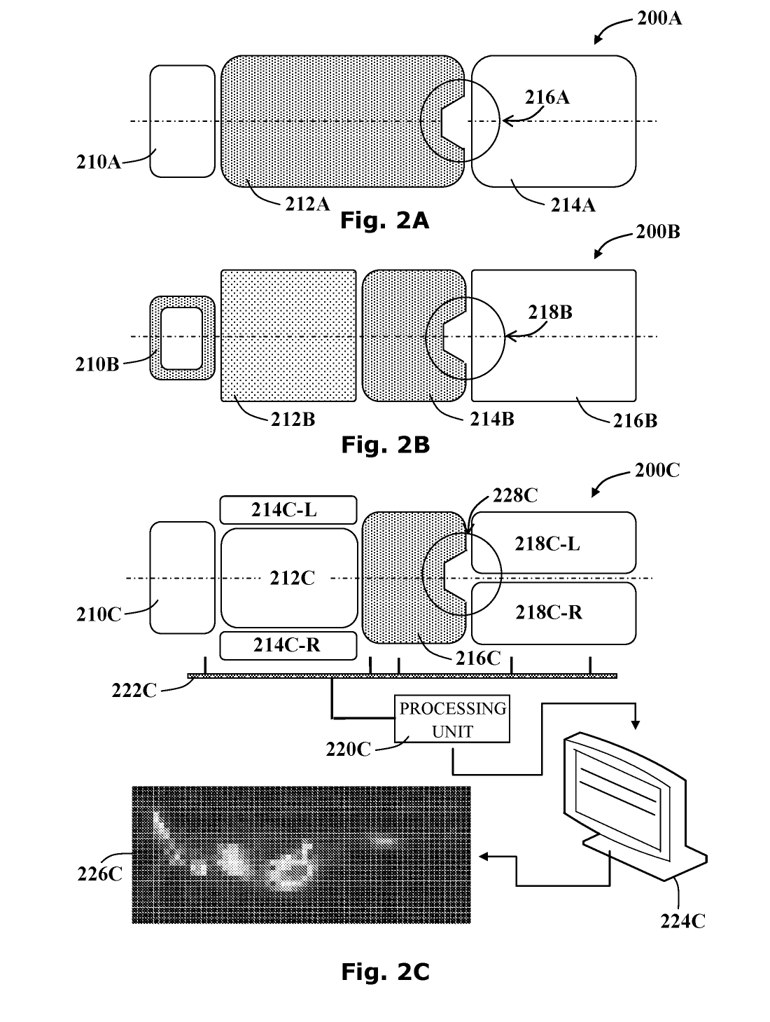

FIG. 2A-C present various possible embodiments of operating theatre tables having different architectures and different number of supporting sections. ‘U’ shaped discontinued sections or hollowed head support, for example and as indicated, is accustomed in operating table architectures, and may prevent continual pressure distribution monitoring. Monitoring pressure distribution during lengthy medical procedures using an operating table is essential for preventing possible development of pressure ulcers, thus this discontinuity requires conductive bridging which is a particular feature of the current disclosure.

Reference is now made to the block diagram of FIG. 2A showing an embodiment of a possible three-section assembly of an operating table 200A.

The operating table 200A may be divided into three sections and includes a head rest section 210A, a back section 212A and a leg section 214A. The back section 212A includes a ‘U’ shaped discontinuity 216A at the lower part of the back section 212A to allow specific medical care according to a medical procedure and possibly additional surgical accessories.

Reference is now made to the block diagram of FIG. 2B showing an embodiment of a possible four-section assembly of an operating table 200B.

The operating table 200B may be divided into four sections and includes a head rest section 210B, a back section 212B, a seat section 214B and a leg section 216B. The head rest section is hollowed to provide comfort and medical aid and the back section 212B includes a ‘U’ shaped notch 218B in the lower part of the back section 212B to allow specific medical care and possibly additional surgical accessories.

It is noted that head positioning is especially important for various surgical postures, as mentioned hereinabove. Extended local pressure on the scalp may lead to localized post-operative alopecia. Thus, the head section may be removable and may have a variety of attachable head rests, geared among other things to reduce pressure.

Reference is now made to the block diagram of FIG. 2C showing an embodiment of a possible multi-section assembly of an operating table 200C connectable to a monitoring system.

The operating table 200C may be divided into multiple sections and includes a head rest section 210C, a back section 212C, a left arm rest 214C-L, a right arm rest 214C-R, a seat section 216C, a left leg section 218C-L and a right leg section 218C-R. Each pressure sensor of a section is connectable to a processing unit 220C via a data bus 222C performing the pressure distribution analysis to be further displayed on a display screen 224C, showing, for example, a normalized pressure distribution of a patient in a lateral posture.

The seat section 216C includes a ‘U’ shaped discontinuity 228C in the lower part of the seat section 216C to allow specific medical care applicable to a medical procedure.

### Flexible Conductive Materials:

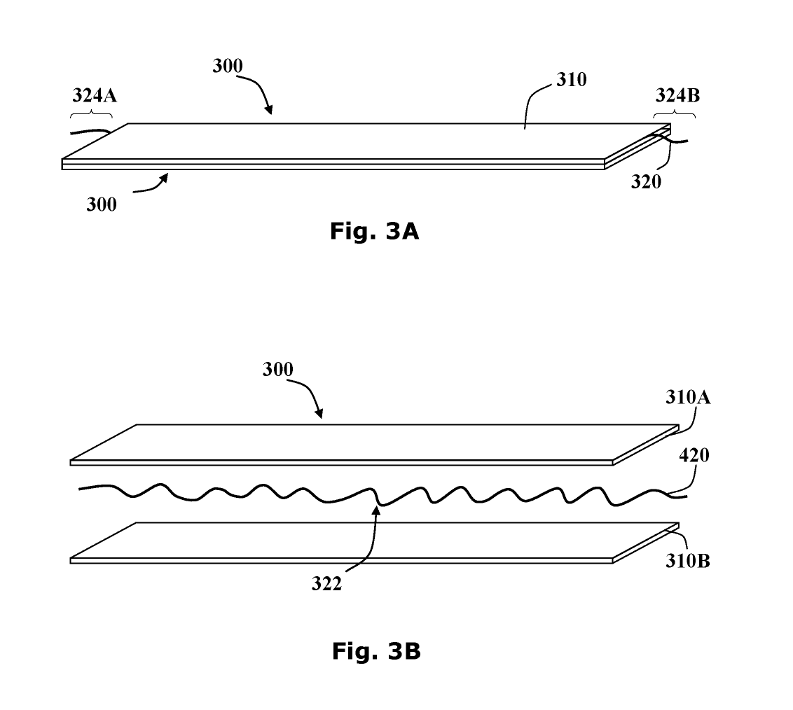

Referring now to FIGS. 3A and 3B another example of a flexible conductive material is presented. In particular FIG. 3A is an isometric view of a composite flexible conductive material 300. The composite flexible conductive material 300 includes a conductive wire 320 embedded into a flexible host material 310. Various materials may be used for the conductive wire such as stainless steel, copper, gold, silver, aluminum, carbon, or the like. Where required, semiconducting material may be used in combination or alternatively to the conducting wire.

Where required, the conducting wire 320 may extend from the ends of the host material 310. The extending sections 324A, 324B of the conducting wire 320 may provide a conducting terminus which may facilitate conductive coupling of the conductive flexible material with connecting wires and other electrical elements.

FIG. 3B shows an exploded view of the composite flexible conductive material 300 illustrating how the host material 310 (FIG. 3A), may be two sheets of laminate material 310A, 310B, such as plastic films or the like, between which the conducting wire 320 is sandwiched. The laminate material may be assembled for example using heat, pressure adhesives, welding or the like.

It is a particular feature of this embodiment that the conducting wire 320 has a length significantly in excess of the length of the host material 310. Accordingly, the conducting wire 320 adopts a sinuous, or wavy, configuration consisting of multiple turns 322 to and fro along the plane of the host material.

The sinuous configuration of the conducting wire 320 may allow the flexible host material to twist, turn, stretch or otherwise reconfigure without being impeded by the mechanical properties of the conducting material of the wire. Accordingly, the elasticity, flexibility, plasticity and other mechanical properties of the composite flexible conductive material 300 may be determined by the flexible host 310 while the electrical properties may be determined by the embedded conducting wire 320.

It is noted that the electrical characteristics of the composite flexible conductive material may be further enhanced by embedding other conducting elements into the host material.

For example, a conducting wire for connecting may be embedded in the flexible material by laminating the conducting wire with a flexible laminate such as plastic film or the like. Lamination may be applied using a variety of methods such as thermal assembly, pressure assembly, adhesive assembly, welding, riveting, heat binding and the like, as well as combinations thereof.

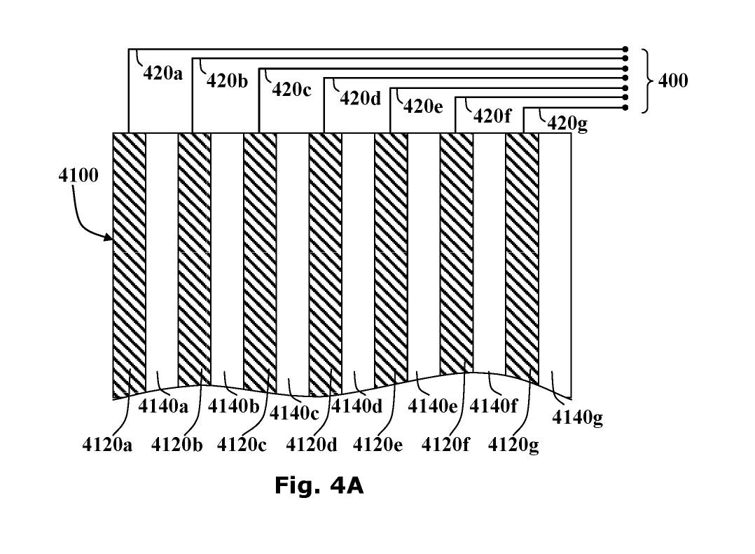

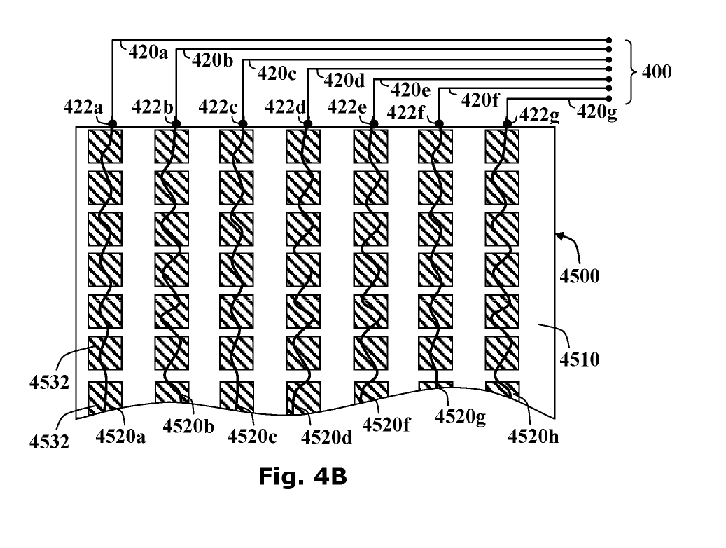

Reference is now made to FIGS. 4A and 4B which schematically represent possible embodiments of the flexible conductive materials 4100, 4500 of the disclosure configured and wired to provide flexible strip electrodes, such as described in the applicant's international application PCT/M2013/059499 which is incorporated herein by reference. Such strip electrodes may be used, for example, in pressure sensing mats or the like such as described hereinafter.

Where such strip electrodes require individual control, a bundle 400 of electrical connecting lines 420 may provide a dedicated conductive path to for each electrode. Various electrical coupling configurations are described herein, although other coupling methods may occur to those skilled in the art.

With particular reference to FIG. 4A, a schematic representation is shown of a segment of selectively conducting fabric 100 wired to function as an array of strip electrodes 4120a-g for use as capacitive plates for example. The electrodes 4120a-g are regions of conducting cloth with intermediate regions 4140a-g forming inter-electrode insulators. The electrode regions 4120 may, for example, comprise material having a high affinity to conductive impregnation, such as polyester yarns, and the inter-electrode insulators 4140 may comprise a material having a low affinity to conductive impregnation, such as nylon. Following electroless plating, the polyester yarns may be impregnated with conductive material whereas the nylon yarns may not be impregnated. The resulting cloth includes an array of conductive electrode strips 4120a-g electrically insulated from each other by insulating inter-electrode regions 4140a-g.

The electrode array 4120 may be wired via a bundle 400 of electric connecting lines 420a-g, thereby providing a dedicated conductive path to each electrode 4120a-g. This dedicated path allows each electrode to be individually controlled or monitored. For example the potential, voltage, current flowing therethrough or the like may be measured and recorded for each electrode individually via a dedicated signal line. It will be appreciated that the electrical connecting lines 420a-g may be conducting wires, ribbons, flatband cables, cables or the like in conductive contact with the electrodes 4120a-g of the fabric. Alternatively or additionally, at least some of the connecting lines 420a-g may comprise conductive fabric sewn, woven or otherwise connected in conductive contact with the electrodes 4120a-g. Indeed, where applicable, the connecting lines 420a-g may also comprise yarns with high affinity to conductive impregnations which are themselves woven, knitted or otherwise incorporated into the selectively conducting fabric 4100 together with the electrodes 4120a-g during production.

Referring now to FIG. 4B which schematically represents a composite flexible conductive material 4500 in which an array of conducting segments 4532 are connected via connecting wires 4520a-h (collectively 4520) to function as an array of strip electrodes and are embedded in a host material 4510.

Each of the connecting wires 4520a-g may be connected to the electric connecting lines 420a-g via a conductive fastening 422a-g. It is particularly noted that where the connecting wires 4520 protrude from the edges of the host material 5100 the conductive fastenings 422a-g may readily connect the extending section of the connecting wires 4520a-g.

Various conductive fastenings may be used to conductively and mechanically couple the connecting wires 4520 to the connecting lines 420a-g. By way of illustration only, a selection of possible conductive fastenings is presented herein below. It will be appreciated that other fastenings may be used where required.

### Utilization of Conductive Materials:

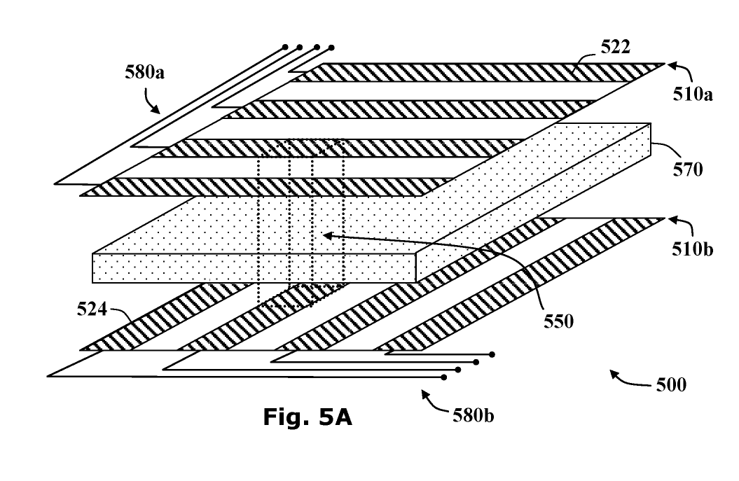

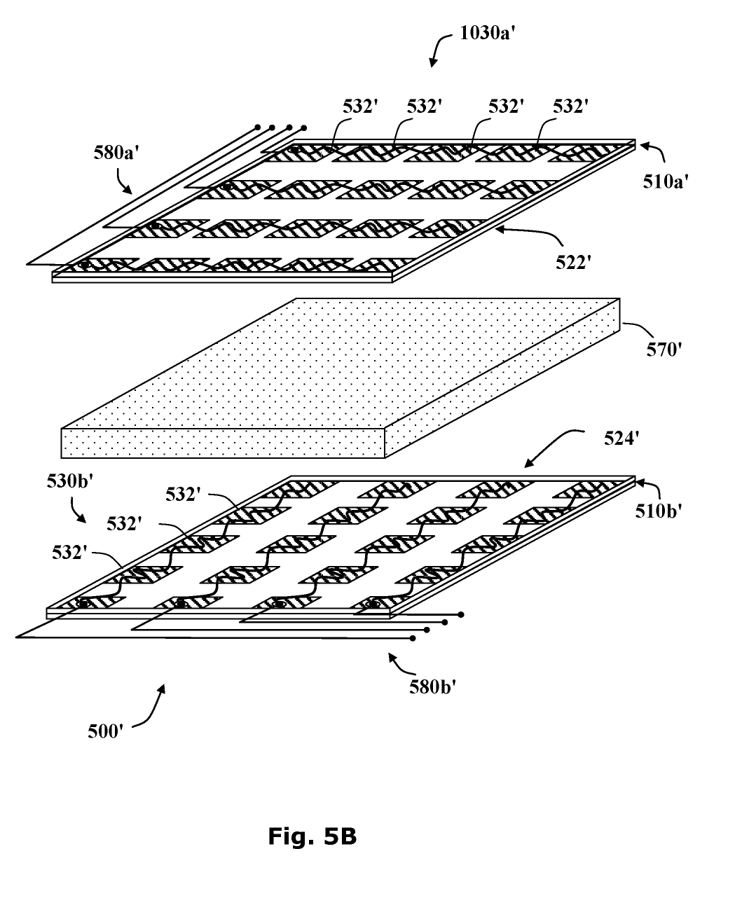

Referring now to FIGS. 5A and 5B, a particular application of the disclosure is schematically represented to illustrate one possible utility of flexible conductive materials such as described herein.

FIG. 5A is an exploded schematic isometric projection of a pressure-detection mat 500 comprising a plurality of pressure sensors 550 arranged in a form of a matrix. The mat 500 includes two layers 510a, 510b of selectively conductive fabric separated by an insulating layer 570 of isolating material. The two layers of selectively conductive fabric 510a, 510b may each include an array of strip electrodes 522, 524 in conductive communication with electrical connecting lines 580a, 580b such as described herein. The two layers of selectively conductive fabric 510a, 510b may be arranged orthogonally. The connecting lines 580a, 580b may be wired to a control unit.

Each pressure sensor 550 may be formed at an overlapping section of the electrode strips 522, 524 at each intersection of a conductive strip with an orthogonal conductive strip. These pressure sensors may be configured such that pressing anywhere on their surface changes the spacing between the two conductive layers, and consequently the capacitance of the intersection. A driving unit may selectively provide an electric potential to the vertical strip 524 and the electrical potential may be monitored on the horizontal strip 522, or vice versa, such that the capacitance of the overlapping section may be determined.

FIG. 5B is an exploded schematic isometric projection of, an alternative embodiment of a pressure-detection mat 500′. Here, the mat 500′ includes two layers 510a′, 510b′ of composite flexible conductive material such as described herein, separated by an insulating layer 570′ of isolating material. The two layers of composite flexible conductive material 510a′, 510b′ may each include an array 530a′, 530b′ of conductive elements 532′ connected via sinuous connecting wires 520′ and embedded in a flexible laminate. The arrays 530a′, 530b′ are configured to form two orthogonal arrays of strip electrodes 522′, 524′ in conductive communication, possibly via conductive riveted fasteners, with electrical connecting lines 580a′, 580b′ such as described herein. The connecting lines 580a′, 580b′ may be wired to a control unit.

It is noted that by providing an oscillating electric potential across each sensor and monitoring the alternating current produced thereby, the impedance of the intersection may be calculated and the capacitance of the intersection determined. The alternating current varies with the potential across a capacitor according to the formula:

I_{ac}=2πfCV_{ac}

where I_{ac} is the root mean squared value of the alternating current, V_{ac} is the root mean squared value of the oscillating potential across the capacitor, f is the frequency of the oscillating potential and C is the capacitance of the capacitor.

Preferably a capacitance sensor will retain its functionality even if it is fully pressed continuously for long periods such as or even longer than 30 days, and keep its characteristics for periods over the lifetime of the sensing mat which is typically more than a year. Notably, the sensor characteristics should preferably be consistent between two separate events.

According to some embodiments, the mat may further include additional sensors configured to monitor additional factors, particularly additional factors influencing the development of bedsores, such as temperature, humidity, moisture, or the like. Such additional sensors may be configured to monitor the factors continuously or intermittently as appropriate to detect high risk combinations of factors. Such measurements may be recorded and stored in a database for further analysis.

Optionally, additional sensors may be located apart from the pressure-detection mat. For example, the mat could be integrated into a seat of a chair and a touch sensor could be integrated into a chair's back support. Where required, additional sensors may be formed from selectively conducting material.

Selectively conductive materials, such as described herein, may be particularly advantageous to such pressure-detection mats because they are flexible. The isolating and insulating layer 570 material may be a compressible, typically sponge-like, airy or poriferous material (e.g. foam), allowing for a significant change in density when pressure is applied to it. Materials comprising the sensing mat are typically durable enough to be resistant to normal wear-and-tear of daily use. Furthermore, the sensing mat may be configured so as not to create false pressure readings, for example when the mat is folded.

Accordingly, the pressure-detection mat 500, 500′ or sensing-mat, may be placed underneath or otherwise integrated with other material layers such as used in standard bed sheets. It will be appreciated that such additional materials may confer further properties as may be required for a particular application. Where required, the conductive material of the selectively conducting fabric may be further covered with an isolating, washable, water resistant, breathing cover mat, allowing minimum discomfort to the subject resting on the mat.

Accordingly the selectively conductive textile may be used to provide a pressure detection mat such as described in the applicant's international patent applications PCT/IL2012/000294, PCT/IB2011/051016, PCT/IB2011/054773 and PCT/IB2012/050829 which are incorporated herein by reference. Such a pressure detection mat may be used to prevent the development of pressure bedsores, decubitus ulcers and the like in subjects by providing indications prompting pressure relieving action being taken. At least one layer of an insulating material 570, 570′ may be sandwiched between a first electrode layer 510a, 510a′ of the selectively conductive textile and a second electrode layer 510b, 510b′ of the conductive textile, wherein the strip electrodes 522, 522′ of the first layer and the strip electrodes 524, 524′ of the second layer overlap at a plurality of intersections. A driving unit (not shown) may be configured to supply electrical potential selectively to the conducting strips 522, 522′ of the first layer 510a, 510a′ via electrical connectors 580a, 580a′ and a control unit (not shown) may be wired to the conductive strips 524, 524′ of the second layer 510b, 510b′ via electrical connectors 580b, 580b′ and operable to control the driving unit. A processor configured to monitor electrical potential on the conductive strips 524, 524′ of the second layer 510b, 510b′, to calculate impedance values for each intersection and to determine pressure applied to the intersection may be provided. Accordingly indications of pressure distribution may be displayed to at least one caregiver, for example on a visual display, such that the caregiver may take pressure relieving action upon the subject.

### Discontinuity Bridging:

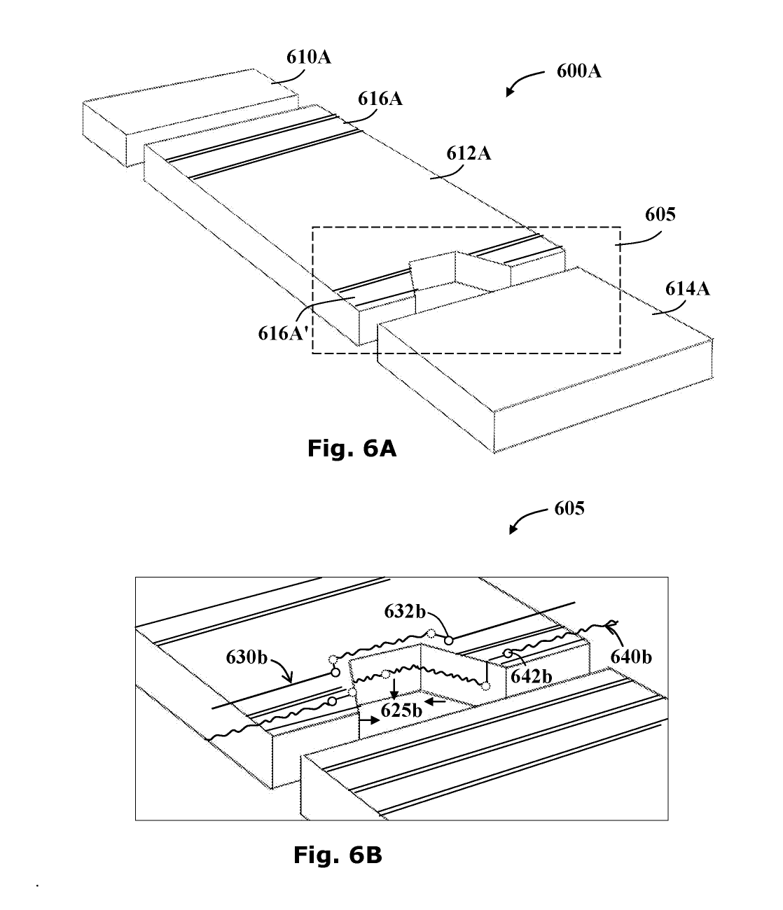

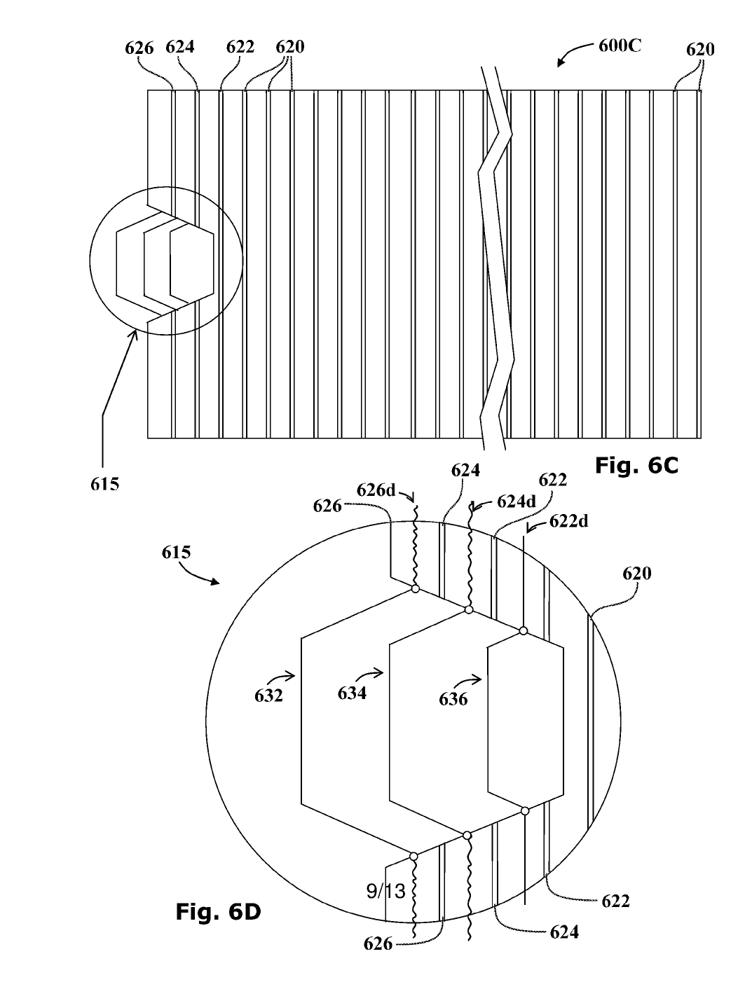

Reference is now made to the block diagram of FIG. 6A showing an isometric embodiment of a three-section anti-decubitus ulcer operating table mattress system 600A for use in an operating table with a discontinuity region 605.

The operating table mattress system 600A may be divided into three sections and includes a head rest section 610A, a body section 612A and a feet section 614A. The body section 612A includes a ‘U’ shaped discontinuity bridging region 605 in the lower part of the body section 612A to allow specific medical care and possibly additional surgical accessories, as required per a specific medical procedure. The ‘U’ shaped discontinuity region 605 disrupts the sensors' connectivity as described in FIG. 5A (sensor 1050), disabling the lower part of the body section to provide proper pressure distribution monitoring. The enlargement 605 provides a detailed view of the technical solution provided for the discontinuity region bridging of the current disclosure.

It is noted that the upper flexible layer comprising a set of conducting strips 616A, spaced at an interval and at least one discontinued conducting strip pair 616A′ to allow the required shape of the pressure detection mat according to required medical procedure/medical instrumentations.

It is further noted that the operation table padding may be manufactured of a composite fabric, such as described in FIG. 5A-B, that allows for real-time pressure-detection and monitoring of possible bedsores development during a medical procedure in an operating theatre table.

It is also noted that the anti-decubitus operating table mattress system 600A is configured to connect to a monitoring system as described in FIG. 1.

Reference is now made to the block diagram of FIG. 6B showing an enlargement embodiment of a discontinuity region bridging 605.

The ‘U’ shaped discontinuity region bridging 605 which is a part of an anti-decubitus operating table mattress system 600A (FIG. 6A) may include a ‘U’ shaped discontinuity region facets 625b, a conducting discontinued strip pair 616A′, a set of bridging connector 630b configured to connect the pair of the conducting discontinued strip pair 616A′ to form a continuous pressure detection region around the discontinuity. Optionally, it is noted that the connecting bridging wires, such as the bridging connector 630b may be a straight cable, optionally connectable via an insulated fastener 632b. Optionally, the connecting bridging wire may be curly, such as demonstrated by the connecting bridging wire 640b.

It is further noted that the connecting bridging wires may be coupled to the upper/lower surface of the flexible insulating material in between the upper and lower conducting fabrics, optionally using insulated fasteners 642b. Optionally, the connecting bridging wires may be attached to ‘U’ shaped discontinuity region facets 625b. Additionally or alternatively, the bridging wires may be kept in a manner to circumvent the discontinuity, as described hereinafter in FIG. 6C-D.

Reference is now made to the block diagram of FIG. 6C showing a top view of an embodiment of a pressure-detection mat with bridging wires over a discontinuity 600C.

The top view of the pressure-detection mat embodiment includes a flexible conductive material having a plurality of conducting strips 620 and discontinued conducting strips 622, 624 and 626 forming a discontinuity with a technical solution using a set of bridging connecting wires, as detailed in detail view 615.

Reference is now made to the block diagram of FIG. 6C showing a detail view 615 providing technical solution using bridging wires over a discontinuity 600.

The detail view 615 of the technical solution with bridging wires over a discontinuity 600C includes a set of conducting strips 622, 624 and 626 each connected with a bridging wire 632, 634 and 636 to form a pressure-detection mat that may circumvent the discontinuity and further connected to a controller of a processing unit (see FIG. 1) via a set of connecting wires 622d, 624d and 626d to provide an operating table monitoring system.

### Visual Monitoring System:

The visual monitoring may provide an indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs portion is orientated at an angle of +15 degrees to the horizontal. Such indication may be presented as a text string, as a map or by way of an icon such as shown in the illustrative display hereinafter.

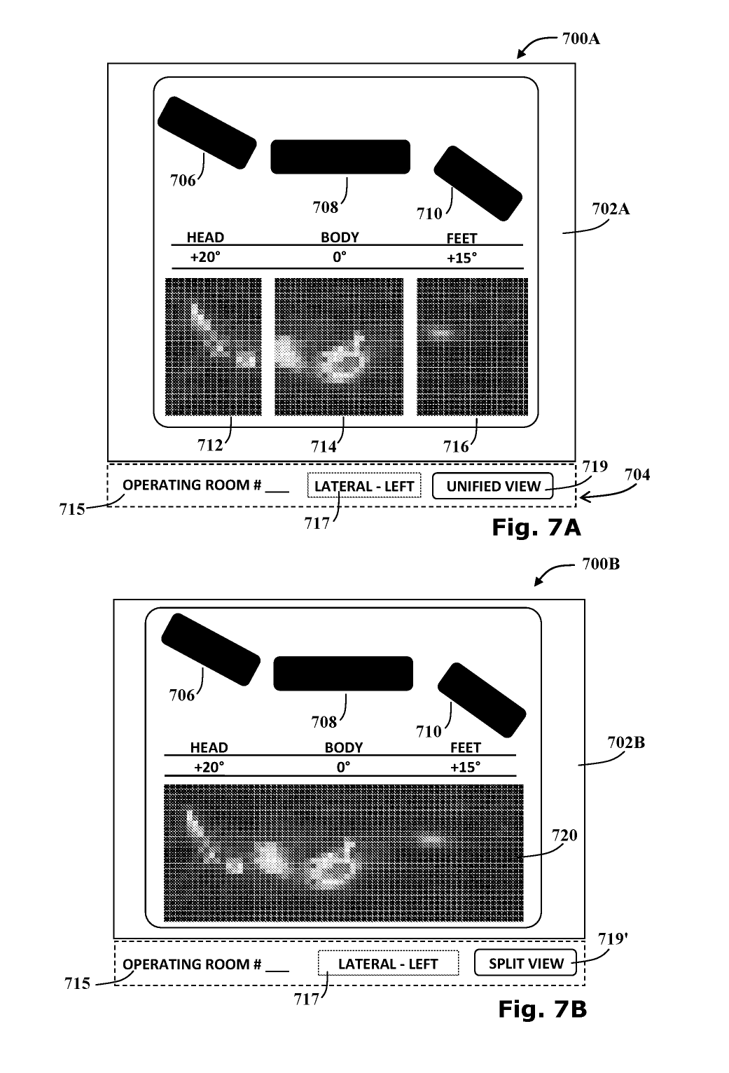

Reference is now made to FIG. 7A-B showing a possible visual presentation system 700A monitoring a subject lying on a pressure-detection mattress of an operating table. The system 700A allows toggling functionality between various visual presentations, possibly combined with suggested orientation changes.

It is noted that the display configurations shown in FIG. 7A-B may apply to a screen display 170 (FIG. 1A) monitoring a pressure-detection sub-system. Optionally or alternatively, the display configuration may be integrated into a central screen display of an operating theatre used for a central monitoring of various medical parameters including subject's pressure-detection.

It is further noted that the display configurations detailed herein are described by way of example and may not be limited to the arrangement of the components or methods set forth in the description or illustrated in the drawings and examples. The various displays and methods of the disclosure may be capable of other embodiments or of being practiced or carried out in various ways.

Alternative methods and display configurations similar or equivalent to those described herein may be used in the practice or testing of embodiments of the disclosure. Nevertheless, particular methods and materials are described herein for illustrative purposes only. The materials, methods, and examples are not intended to be necessarily limiting.

Referring to FIG. 7A, showing a split view representation of a screen display 700A providing current orientation and pressure distribution of a three-section system supporting of subject's body—head, body and feet. This may be applicable, for example, where the subject is lying over three sections of an adjustable pressure distribution surface, such as a three-sectioned mattress of an operating table. It is further noted that various operating table platforms may have fewer or a greater number of pressure detection sections, possibly depending upon the type of surgical procedure, adding multiple sensing sections for monitoring pressure distribution such as arm rests, back rests, head rests and the like.

The screen display 700A may include a display frame 702A with an upper viewing frame displaying the orientation for each section of the adjustable pressure-detection surface 706, 708 and 710, and a lower viewing frame displaying the pressure distribution map for each section of the adjustable pressure-detection surface 712, 714 and 716.

Additionally or alternatively, the upper viewing frame of the screen display 700A may provide orientation indication of each monitored section of the subject's body. For example, a user interface may indicate that the head portion is orientated at an angle of +20 degrees to the horizontal, the body portion is orientated at an angle of 0 degrees to the horizontal, and the lower limbs portion is orientated at an angle of +15 degrees to the horizontal. Such indication may be presented as a text string or may be displayed graphically as a map, an icon or as may otherwise occur to those skilled in the art.

Optionally or additionally, the screen display 700A may include a control panel 704, displaying associated information such as operating room number 715, body posture indication 717 such as Trendelenburg, Lateral, Prone, Supine and the like and a toggling button to allow various views, for example, selecting to view a body unified view, body split view and the like. Optionally the control panel may include additional control functionality such as timer-based re-positioning suggestion, alarming if pressure is detected to exceed a pre-configured threshold value and the like.

It is particularly noted that the monitoring system supports normalizing of display when viewed as successive split views of sections or when viewed as a unified pressure distribution map of the whole mattress.

It may be noted that the current display may be continually updated with the changes of actual orientation values for each surface section, or may be updated at a configurable time interval.

FIG. 7B is showing a unified view representation of a screen display 700B providing current orientation and pressure distribution of a three-section system supporting of subject's body—head, body and feet, after pressing the toggling button 719 of FIG. 7A. The screen display 700B includes a toggling button 719′ allowing to return to a split view, as shown in FIG. 7A.

It is noted that the toggling button may provide additional alternatives, such as view a specific section of head rest, back rest and the like. Similarly, for multi-section systems, additional specific pressure distribution may exist such as left/right arm rest, left/right limb rest, seat rest and the like.

Optionally or additionally, the screen display 300C may be used as part of a central operating theatre control system, with additional control panel to allow switching from monitoring one operating theater to monitoring another operating theatre.

### Posture Visual Samplings:

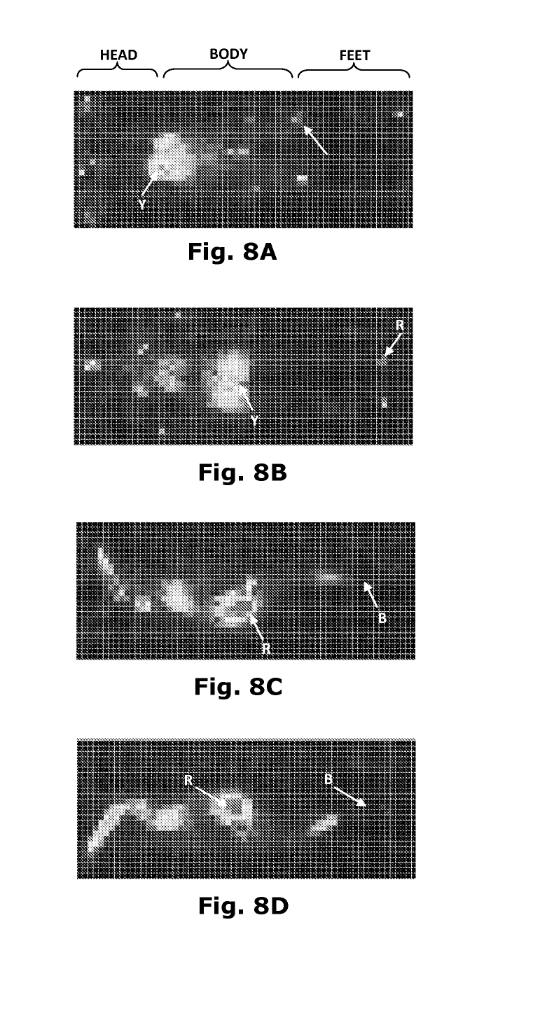

Reference is now made to the pressure distribution maps of FIG. 8A-D, showing various representations of how pressure data may be displayed on a display screen 170 (FIG. 1) of the monitoring system 100 (FIG. 1) for an adjustable single section of an operating table, or a complete normalized pressure distribution view based upon data recorded by at least one pressure-detection mat. Respectively, FIG. 8A-D show the pressure distribution for a subject lying on his abdomen (FIG. 8A), his back (FIG. 8B), his left side (FIG. 8C) and his right side (FIG. 8D).

The display system 170 (FIG. 1) may be a computer in communication with the data storage unit 160 (FIG. 1), for example. Each display screen may show a matrix of pixels, each pixel may represent one sensor of the pressure-detection sheet. The pressure detected by each pixel may be represented by a visual indication. A grayscale may be used such that higher pressures are indicated by different shades, darker grays, for example. Alternatively or additionally, colors may be used, for example, indicating high pressure formed between a subject's body and the surface on which the subject rests by displaying the pixel in a distinctive color, such as red (marked with R). Likewise, pixels representing sensors which detect low pressure or no pressure at all may be presented in other colors such as yellow (marked with Y), blue (marked with B) or black. It is understood that other colors or combinations are contemplated for the display screen 170 (FIG. 1). Furthermore, the ability to normalize the pressure scale displayed is contemplated, such as for allowing pressure readings to be scaled up or down depending on the surface the subject is lying on. Such a feature may be useful, for example, to ensure that a caregiver is still alerted to body areas experiencing relatively high pressure even when the patient is lying on an airbed that lowers absolute pressure.

### Bridging Method:

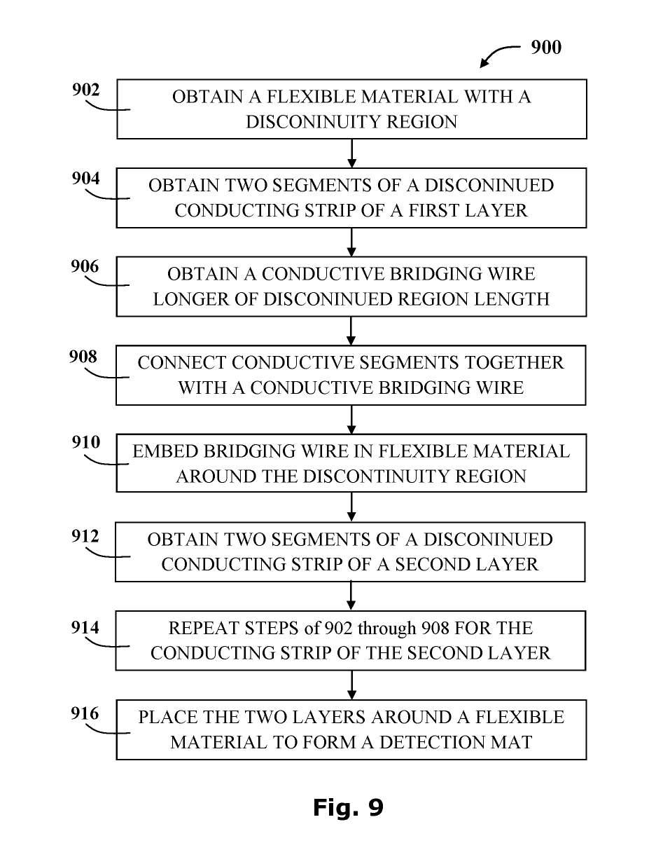

Referring now to the flowchart of FIG. 9, selected actions of a method for making a flexible pressure detection mat having a discontinuity region to allow pressure detection around the discontinuity by providing a technical solution of bridging, as a particular feature of the current invention.

The method may include: obtaining a flexible material with a discontinuity—step 902, where the discontinuity may have an open shape contour or a closed shape contour; then for the first layer, obtaining two conducting segments of a discontinued conducting strip of a first layer—step 904; obtaining a conductive bridging wire longer than the length of the discontinuity region—step 906; connecting the conducting segment together with the conductive bridging wire—step 908; embedding the conducting bridging wire in the flexible material around the region discontinuity—step 910. For the second layer, obtaining two conducting segments of a discontinued conducting strip of a second layer—step 912; and repeating the steps of 906 through 910 for the second layer segments—step 914; then placing the two layers around the flexible material to form a pressure-detection mat—step 916.

It is noted that the bridging wires, for example, may be embedded and coupled to the flexible insulating material by an insulated fastener such that they are not exposed, thus avoiding any disturbance with required medical instruments that may be used in a surgery procedure. As appropriate, the above-described example is presented for illustrative purposes only, still further bridging connecting wires for connecting the conducting strips may be kept outside the covering layers.

For example, conducting wire may be embedded and coupled to the flexible insulating material by laminating the bridging wires with a flexible laminate such as plastic film or the like. Lamination may be applied using a variety of methods such as thermal assembly, pressure assembly, adhesive assembly, welding, riveting, heat binding and the like, as well as combinations thereof.

### Normalizing Method:

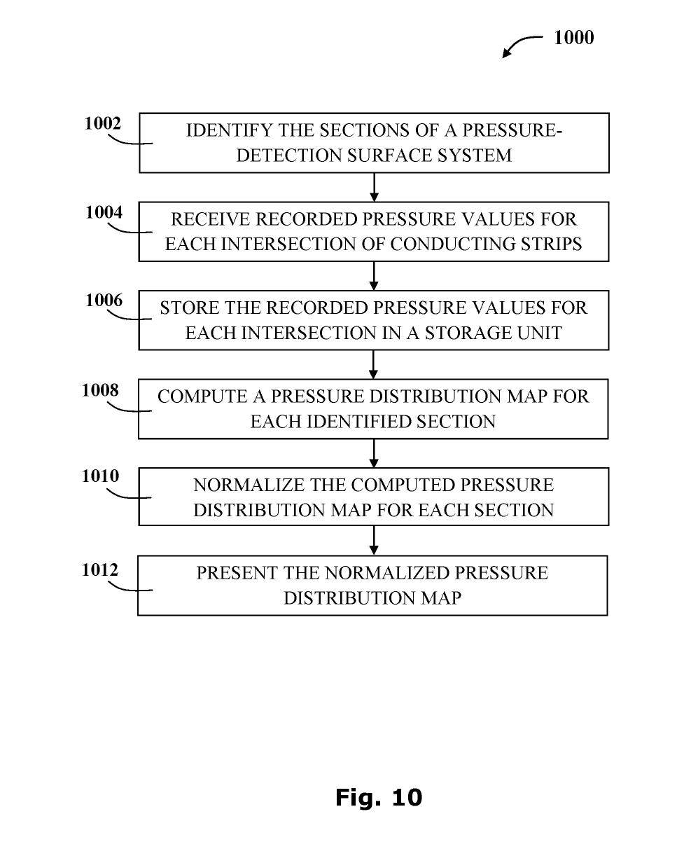

Referring now to the flowchart of FIG. 10, selected actions of a method for normalizing pressure distribution map of a subject lying on an operation table is presented as a particular feature of the current invention.

The method may include: identifying the surface sections of the pressure-detection surface system forming the upper side of the operating table—step 1002, where at least one section comprises a discontinuity contour; receiving pressure recorded data values monitored for each of the intersections for a surface section—step 1004; storing the data recorded of pressure values for each intersection in at least one data storage of a storage unit—step 1006; computing a pressure distribution map for each identified surface section based upon the stored pressure values received—step 1008; normalizing the computed pressure distribution map for each of the identified surface sections—step 1010; and presenting a normalized pressure distribution view comprising at least one pressure distribution map on at least one display unit.

It is noted that a processor may be configured to monitor electrical potential on the conductive strips (See FIG. 5A), further to calculate impedance values for each intersection, thus to determine pressure applied to the intersection may be provided.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially” of means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A pressure-detection mat comprising:
at least one layer of an insulating material sandwiched between a first layer of conducting strips and a second layer of conducting strips, the first and second layers of conducting strips overlapping at a plurality of intersections;
a first bundle of connecting wires for connecting the first layer of conducting strips to a control unit; and
a second bundle of connecting wires for connecting the second layer of conducting strips to the control unit, at least one conducting strip of the first layer of conducting strips is non-continuous and includes at least a first segment and a second segment, and the first segment is connected to the second segment by a bridging wire to provide conductive communication therebetween.

2. The pressure-detection mat of claim 1, wherein the first segment includes a conductive material and a conductive wire.

3. The pressure-detection mat of claim 1, wherein at least one of the conducting strips in each of the first and second layers of conducting strips includes an array of strip electrodes embedded in the insulating material, each of the strip electrodes in the array of strip electrodes including:
a plurality of segments of conductive material;
a connecting wire in conductive contact with the plurality of segments and adopting a sinuous configuration along the strip electrode; and
a flexible laminate into which the segments and the connecting wire are embedded, the array of strip electrodes overlap at a plurality of intersections.

4. The pressure-detection mat of claim 1, wherein the pressure-detection mat includes a discontinuity having a closed shape within a boundary of the pressure-detection mat.

5. The pressure-detection mat of claim 1, wherein the pressure-detection mat includes a discontinuity shaped as an open shape within a boundary of the pressure-detection mat.

6. The pressure-detection mat of claim 1, wherein the bridging wire is coupled to the insulating material by an insulated fastener.

7. The pressure-detection mat of claim 1, wherein the bridging wire circumvents a discontinuity in the pressure-detection mat.

8. The pressure-detection mat of claim 1, wherein the bridging wire is in conductive contact with the first bundle of connecting wires.

9. A pressure-detection surface system comprising:
a first pressure-detection mat of claim 1;
a driving unit configured to supply electrical potential selectively to the first and second layer of conducting strips, the control unit wired to the first and second layer of conductive strips and operable to control the driving unit;
a processor configured to monitor electrical potential on the first and second layer of conductive strips, to calculate impedance values for each of the intersections and to determine pressure applied to the intersection; and
at least one display unit configured to present a pressure distribution map.

10. The pressure-detection surface system of claim 9, further comprising at least one second pressure-detection mat, and the processor is further configured to normalize the pressure distribution map from the first pressure-detection mat and the second pressure-detection mat, and the at least one display unit is further configured to present a normalized map.

11. A method for manufacturing a pressure detection mat, comprising:
obtaining at least one first layer including a plurality of conducting strips;
obtaining at least one second layer including a plurality of conducting strips;
obtaining at least one layer of an insulating material sandwiched between the first layer and the second layer;
providing at least one non-continuous conducting strip including at least a first segment and at least a second segment; and
connecting the first segment and the second segment of the non-continuous conducting strip with at least one bridging wire.

12. The method of claim 11 further comprising embedding the at least one bridging wire within a flexible laminate into the insulating material.

13. A method for processing pressure distribution in a pressure-detection surface system comprising a first pressure-detection mat, a driving unit configured to supply electrical potential selectively to a first and second layer of conducting strips overlapping at a plurality of intersections, a control unit wired to the conductive strips and operable to control the driving unit, a processor configured to monitor electrical potential on the conductive strips, to calculate impedance values for at least one intersection and to determine pressure applied to the at least one intersection and at least one display unit configured to present a pressure distribution map, the method comprising:
identifying one or more surface sections of the pressure-detection surface system;
receiving pressure values of at least one intersection for each of the one or more surface sections;
storing the pressure values for the at least one intersection for each of the one or more surface sections in at least one data storage;
computing a pressure distribution map for each of the one or more surface sections based upon the pressure values;
normalizing said pressure distribution map for each of the one or more surface sections; and
presenting a normalized pressure distribution view comprising at least one pressure distribution map on at least one display unit.

14. The method of claim 13, wherein said first pressure-detection mat comprises:
at least one layer of an insulating material sandwiched between the first layer of conducting strips and the second layer of conducting strips;
a first bundle of connecting wires for connecting the conducting strips of the first layer to a control unit; and
a second bundle of connecting wires for connecting the conducting strips of the second layer to said control unit, the pressure-detection mat includes at least one discontinuity such that at least one conducting strip of the first layer is non-continuous and includes at least a first segment and a second segment, the first segment is connected to the second segment by a bridging wire to provide conductive communication therebetween.
